(19) United States
(12) Patent Application Publication
(10) Pub. No.: US 2014/0171674 A1
(43) Pub. Date: Jun. 19, 2014

(54) NUTRITIONAL COMPOSITIONS COMPRISING HIGH OLEIC ACID CANOLA OIL

(71) Applicant: University of Manitoba, Winnipeg (CA)

(72) Inventors: Thomas NETTICADAN, Winnipeg (CA); Carla TAYLOR, Winnipeg (CA); Sijo Joseph THANDAPILLY, Winnipeg (CA)

(73) Assignee: University of Manitoba, Winnipeg (CA)

(21) Appl. No.: 14/132,841

(22) Filed: Dec. 18, 2013

Related U.S. Application Data
(60) Provisional application No. 61/738,932, filed on Dec. 18, 2012.

(51) Int. Cl. A23L 1/30; A61K 31/202

(52) U.S. Cl. CPC A23L 1/3008; A61K 31/202
USPC 554/224

(57) ABSTRACT

A dietary regimen for mitigating cardiovascular disease risk in a subject, the regimen comprising consumption of at least one of a functional food composition comprising high oleic canola oil, a dietary supplement comprising high oleic canola oil, and a nutraceutical composition comprising high oleic canola oil, whereby the subject consumes at least 14 mg/100 g of body weight every 24-hour diurnal period.

### FIELD OF THE DISCLOSURE

The present disclosure relates to compositions comprising high oleic acid canola oil for use to reduce development of cardiovascular disease risk factors and for mitigating cardiovascular disease risk.

### BACKGROUND OF THE DISCLOSURE

Obesity is one of the most prevalent nutritional disorders in the populations of most developed countries. According to the World Health Organization (WHO), the prevalence of obese individuals has risen dramatically during the last three decades to near epidemic proportions. Recent WHO surveys indicate that more than 1 billion adults in the world are overweight with close to 1 million of these considered to be clinically obese.

Obesity is associated with physiological changes that cause or contribute to a wide variety of metabolic diseases, including Type 2 diabetes, hypertension and coronary artery disease. Because of its deleterious effects on various cardiovascular disease (CVD) risk factors and its adverse effects on cardiac structure and function, obesity is a major contributor to CVD and a major cause of death.

Current strategies for combating obesity and its associated complications generally focus on advocating consumption of relatively low-fat diets combined with increased physical activity. Over-the-counter and prescription therapeutic agents are commonly used to: (i) repress appetites to reduce food intake, or (ii) increase thermogenesis, or (iii) suppress the body's absorption of fats, or (iv) inhibit the differentiation of adipocytes. Other therapeutic agents include anoretics exemplified by sibutramine and combinations of phentermine-fenfluramine. However, such therapies are often not successful in combating obesity. Other thereapy strategies include administration of medicaments that specifically target CVD risk factors, such as those exemplified by lipid-lowering drugs, anti-hypertensive medications, anti diabetic agents and the like. However, these types of therapies often result in serious side effects, including adverse effects on the central nervous system, and the development of hypertension.

### SUMMARY OF THE DISCLOSURE

The present disclosure pertains to dietary regimens for reducing the onset and development of CVD risk factors, and for mitigating physiological risks associated CVD, wherein the dietary regimens comprise regular consumption of compositions comprising high oleic acid canola oil.

One embodiment of the present disclosure pertains to foodstuff compositions comprising high oleic acid canola, wherein the foodstuff compositions are formulated for regular routine consumption by a subject. The foodstuff compositions may comprise drink compositions, and/or emulsions exemplified by salad dressings, and/or baked goods and/or deep-fried goods, and the like. Some aspects relate to use of the foodstuff compositions on a regular routine basis for reducing the onset and development of CVD risk factors, and for mitigating physiological risks associated CVD.

Another embodiment of the present disclosure pertains to functional food compositions comprising high oleic acid canola, wherein the functional foodstuff compositions are formulated for regular routine consumption by a subject. The functional food compositions may comprise drink compositions, and/or emulsions exemplified by salad dressings, and/or baked goods and/or deep-fried goods, and the like. Some aspects relate to use of the functional food compositions on a regular routine basis for reducing the onset and development of CVD risk factors, and for mitigating physiological risks associated CVD.

Another embodiment of the present disclosure pertains to dietary supplements comprising high oleic acid canola oil, wherein the dietary supplements are formulated for regular routine consumption by a subject. The dietary supplements may be formulated into drink compositions or alternatively, into fluid emulsions, or alternatively into capsules, and the like. Some aspects relate to use of the dietary supplements on a regular routine basis for reducing the onset and development of CVD risk factors, and for mitigating physiological risks associated CVD.

Another embodiment of the present disclosure pertains to nutraceutical compositions comprising high oleic acid canola oil, wherein the nutraceutical compositions are formulated for regular routine consumption by a subject. The nutraceutical compositions may be formulated into fluid emulsions, or alternatively into capsules, and the like. Some aspects relate to use of the dietary supplements on a regular routine basis for reducing the onset and development of CVD risk factors, and for mitigating physiological risks associated CVD.

### BRIEF DESCRIPTION OF THE DRAWINGS

The features of the present disclosure will become more apparent from the following description in which reference is made to the appended drawings wherein:

FIGS. 1(A)-1(D) are charts showing the effects of different dietary oils in 12-wk diets on: 1(A) total feed consumed, (1(B) weight gain during the 12-wk period, 1(C) final body weight at the end of the 12-wk period, and 1(D) the feed efficiency ratio (i.e., the total weight gain in grams/total feed intake in grams) during the 12-wk period. Statistical differences among means (p<0.05) are indicated by different lower case letters. An absence of letters indicates that means are not statistically different;

FIGS. 2(A)-2(D) are charts showing the effects of different dietary oils in 12-wk diets on adiosity at the end of the 12-wk period, on: 2(A) mesenteric fat, 2(B) epididymal fat, 2(C) peri-renal fat, and 2(D) visceral fat (visceral fat includes mesenteric fat pads, epididymal fat pads, and peri-renal fat pads). Statistical differences among means (p<0.05) are indicated by different lower case letters. An absence of letters indicates that means are not statistically different;

FIG. 3 is a chart showing the effects of 12-week high-fat dietary regimens comprising different types of oils, on the heart weight to tibia length ratios of obese-prone rats;

FIG. 4 is a chart showing the effects of 12-week high-fat dietary regimens comprising different types of oils, on the cardiac ejection fractions of obese-prone rats;

FIG. 5 is a chart showing the effects of 12-week high-fat dietary regimens comprising different types of oils, on the isovolumic relaxation time (IVRt) of obese-prone rats;

FIG. 6 is a chart comparing the effects on the heart weight to tibia length ratios of obese-prone rats fed a high-fat diet regimen comprising high oleic acid canola oil with the heart weight to tibia length ratios of Sprague-Dawley rats fed a low-fat diet regimen;

FIG. 7 is a chart comparing the effects on isovolumic relaxation time of obese-prone rats fed a high-fat diet regimen comprising high oleic acid canola oil with the isovolumic relaxation time of Sprague-Dawley rats fed a low-fat diet regimen;

FIG. 8 is a chart comparing the effects on cardiac ejection fraction of obese-prone rats fed a high-fat diet regimen comprising high oleic acid canola oil with cardiac ejection fraction of Sprague-Dawley rats fed a low-fat diet regimen; and

FIG. 9 is a chart comparing the effects on the cardiac output of obese-prone rats fed a high-fat diet regimen comprising high oleic acid canola oil with the cardiac output of Sprague-Dawley rats fed a low-fat diet regimen.

### DETAILED DESCRIPTION

Unless defined otherwise, all technical and scientific terms used herein have the same meaning as commonly understood by one of ordinary skill in the art to which the disclosure belongs. Certain terms are discussed in the specification to provide additional guidance to the practitioner in describing the methods, compositions and the like of embodiments of the disclosure, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the disclosure herein. Although any methods and materials similar or equivalent to those described herein can be used in the practice or testing of the present disclosure, the preferred methods and materials are now described.

To facilitate understanding of the disclosure, the following definitions are provided.

As used herein, “cardiovascular disease” refers to a class of diseases that involves the circulatory system, including the heart and blood vessels (arteries and veins), whether the blood vessels are affecting the lungs, the brain, kidneys or other parts of the subject's body. Examples of such diseases include, but are not limited to, ischemic heart disease leading to a myocardial infarction, coronary heart disease, cerebrovascular disease (stroke) and atherosclerosis.

As used herein, “cardiovascular disease risk factors” refers to those factors that cause or contribute to the subject's chances of developing cardiovascular disease. Non-limiting examples of cardiovascular disease risk factors include total cholesterol, HDL cholesterol, presence of plaques, percentage body fat, hypertension (high blood pressure), pulmonary hypertension, cardiac dysfunction, among others known to those skilled in this art.

As used herein, “isovolumetric relaxation time” (IVRT) is an interval in the cardiac cycle from the closure of the aortic valve to onset of filling by opening of the mitral valve. IVRT is used as an indicator of diastolic disfunction, specifically a decline in the performance of the left ventricle or both left and right ventricles during the phase of the cardiac cycle with the heart is relaxing and filling with blood ingressing from the inferior vena cava.

As used herein, “reducing development of cardiovascular disease risk factors” refers to reducing or lowering the chance of developing cardiovascular disease risk factors in a subject when compared to the chance of developing cardiovascular disease risk factors in the same subject in the absence of dietary supplements comprising high oleic acid canola oil.

As used herein, “mitigating cardiovascular disease risk” refers to lessening the risk in a subject of developing cardiovascular disease or delaying the onset or progression of cardiovascular disease risk factors that increase the risk in a subject of developing cardiovascular disease.

As used herein, “dietary supplement” refers to a product that contains a “dietary ingredient” intended to supplement the diet. “Dietary ingredient” includes, but is not limited to, vitamins, minerals, fiber, fatty acids, amino acids, herbs or other botanicals, and substances such as enzymes, organ tissues, glandulars, and metabolites. Dietary supplements can also be extracts or concentrates, and may be found in many forms such as, but not limited to, tablets, capsules, softgels, gelcaps, liquids or powders.

As used herein, “functional food composition” refers to a food product that is consumed as part of a regular diet, and has demonstrable benefits for mitigating cardiovascular disease risk.

As used herein, “nutraceutical composition” refers to a product isolated or purified from plant materials, and which is generally provided in formats not usually associated with foods. A nutraceutical composition has demonstrable physiological benefits for protection against chronic disease, such as mitigating cardiovascular disease risk.

As used herein, “nutraceutical carrier” refers a suitable vehicle which is biocompatible and nutraceutically acceptable, and may comprise one or more solid, semi-solid or liquid diluents, excipients, flavours or encapsulating substances which are suitable for consumption.

As used herein, “biocompatible” refers to a compound or mixture of compounds that does not generate a significant undesirable response in a subject for the intended utility. Biocompatible materials are typically non-toxic for the intended utility. For human utility, a biocompatible compound or mixture of compounds is most preferably non-toxic to humans or human tissues.

As used herein, “conventional canola oil” refers to an oil crushed from canola seed, wherein the oil contains comprises: (i) about 61% mono-unsaturated fatty acids exemplified by oleic acid, (ii) about 32% polyunsaturated fatty acids wherein about 11% of the polyunsaturated fatty acids is alpha-linoleic acid and about 21% of the polyunsaturated fatty acids is linoleic acid, and (iii) about 7% saturated fatty acids (Table 1).

As used herein, “high oleic acid canola oil” refers to an oil crushed from seeds produced by high oleic acid canola lines. High oleic acid canola oil, in comparison to conventional canola oil, has: (i) an oleic acid content of about 67% and greater, (ii) about 23% polyunsaturated fatty acids wherein about 3% of the polyunsaturated fatty acids is alpha-linoleic acid and about 20% of the polyunsaturated fatty acids is linoleic acid (Table 1). Oleic acid is a monounsaturated, omega-9 fatty acid. Alpha-linoleic acid is an omega-3 fatty acid and linoleic acid is an omega-6 fatty acid.

TABLE 1
Fatty acid composition of different edible oils

| | Dietary Oils | | | | | | |
|---|---|---|---|---|---|---|---|
| | | | High oleic | | | | |
| | High | | acid canola | | | | |
| | oleic acid | | oil + | Conventional | High | | |
| Fatty Acid | canola | Conventional | conventional | canola oil + | linoleic | Soybean | |
| Composition | oil | canola oil | canola oil | flax oil | safflower oil | oil | Lard |
| SFA* | 7 | 7 | 7 | 7.5 | 10 | 15 | 40.5 |
| C16:0 | 4 | 4 | 4 | 4.4 | 7 | 9 | 22.6 |
| C18:0 | 2 | 2 | 2 | 2.4 | 2 | 4 | 13.1 |
| MUFA** | 70 | 61 | 65 | 51 | 14 | 23 | 44.8 |
| C18:1n9 (OA) | 70 | 61 | 65 | 46 | 14 | 23 | 44.8 |
| PUFA (% Fat)*** | 23 | 32 | 28 | 41 | 76 | 62 | 14.7 |
| C18:2n6 (LA) | 20 | 21 | 21 | 19 | 75 | 54 | 13.1 |
| C18:2n3 (ALA) | 3 | 11 | 7 | 22 | 1 | 8 | 1.6 |
| n6:n3 ratio | 7:1 | 2:1 | 3:1 | 0.8:1 | 75:1 | 7:1 | 8:1 |

*saturated fatty acids
**mono-unsaturated fatty acids
***poly-unsaturated fatty acids

As used herein, “effective amount” refers to an amount of a given compound that achieves a desired effect.

As used herein, “subject” refers to a human and other vertebrate mammalian species and includes for example, but is not limited to primates, cows, pigs, sheep, goats, horses, buffalo, lama, dogs, cats, rabbits, mice, rats hamsters and guinea pigs, or transgenic species thereof.

As used herein, “regular consumption” means consuming one or more of the foodstuff compositions, the functional food compositions, the dietary supplements, and the nutriceutical compositions at least once on a daily basis whereby an amount of at least 14 mg/100 g of a subject's body weight is consumed every 24-hr diurnal cycle.

Obesity predisposes a subject to or alternatively is associated with numerous cardiac complications such as coronary heart disease, heart failure, sudden death due to adverse impacts on the cardiovascular system and other types of cardiovascular disease (“CVD”). In fact, obesity is an independent risk factor for CVD, and CVD risks have been documented in obese children (Poirier, P. et al., 2006, Obesity and Cardiovascular Disease: Pathophysiology, Evaluation, and Effect of Weight Loss. Circulation 113:898-918). It is known that an altered metabolic profile and a number of adverse changes in cardiac structure and function occur in a subject as adipose tissue accumulates in excess amounts (Poirier, P. et al., 2004, Impact of bariatric surgery on cardiac structure, function and clinical manifestations in morbid obesity. Expert Rev. Cardiovasc. Ther. 2:193-201). Therefore, obesity is thought to affect the heart through its influence on various risk factors such as but not limited to, dyslipidemia, hypertension, glucose intolerance, inflammatory markers, obstructive sleep apnea/hypoventilation, the prothrombotic state, and to have adverse affects on cardiac structure and cardiac function.

The inventors have found that regular dietary consumption of foodstuffs comprising high oleic acid canola oil and alternatively, combinations of high oleic acid canola with conventional canola oil, reduces the development of obesity-induced cardiac complications such as the development of CVD risk factors.

Accordingly, the present disclosure pertains to methods for reducing the potential development of cardiovascular disease risk factors and for mitigating cardiovascular disease risk, through the regular use i.e., consumption of compositions comprising high oleic acid canola oil.

Some aspects pertain to consumption of foodstuffs comprising high oleic acid canola oil. Some aspects pertain to consumption of functional foods comprising high oleic acid canola oil. Some aspects pertain to consumption of nutraceuticals comprising high oleic acid canola oil. Some aspects pertain to consumption of dietary supplements comprising high oleic acid canola oil.

The development of various CVD risk factors may be reduced by regular consumption of dietary supplements comprising high oleic acid canola oil as disclosed herein. The consequences of reducing the potential occurrence of CVD risk factors may result in reduced potential, for example, for development of abnormal cardiac function, including without limitation, abnormal isovolumetric relaxation time (“IVRT”) and abnormal ejection fraction, of an at-risk subject. A person skilled in the art will understand that IVRT can be used as an indicator of diastolic dysfunction, a risk factor for CVD, which leads to an impaired relaxation of the ventricles, the pumping chambers of the heart, after contraction. IVRT is an interval in the cardiac cycle, from the closure of the aortic and pulmonic valves (which causes the second heart sound) to the onset of filling of the ventricles by the opening of the atrioventricular valves (mitral and tricuspid) that separate the atria from the ventricles. During this IVRT, the ventricular muscle decreases its tension without lengthening so that ventricular volume remains unaltered. IVRT can be measured using any method known in the art, for example, but not limited to, Doppler echocardiography, M-mode sonography, 2D-guided M-mode echocardiography, or using simultaneous phonocardiogram and transmitral Doppler. A normal IVRT is approximately 70±12 ms, and approximately 10 ms longer in subjects over forty years. IVRTs that are prolonged in length indicate poor myocardial relaxation. These IVRTs are usually in excess of 110 ms.

The ejection fraction of a heart is the amount of blood pumped out of the left ventricle divided by the maximum volume remaining in the left ventricle at the end of diastole or the relaxation phase (i.e., in a filled ventricle). It is measured on the left ventricle (left ventricular ejection fraction, or LVEF) because the left ventricle is the heart's main pumping chamber, pushing oxygen-rich blood to the entire body. A normal ejection fraction is greater than 50%. Systolic heart failure has a decreased ejection fraction of less than 50%.

Other measures of CVD risk factors may include, without limitation, dyslipidemia marked by abnormal concentrations of lipids and lipoproteins in the blood (for example, elevated LDL levels and decreased HDL levels); heart to body weight ratio as a marker for cardiac hypertrophy; abnormal cardiac structure; and hypertension.

In another aspect of the present disclosure, methods for mitigating cardiovascular disease risk in a subject are disclosed wherein the methods comprise regular routine consumption of dietary supplements comprising high oleic acid canola oil and/or combinations of high oleic acid canola oil and conventional canola oil.

In another aspect of the present disclosure, methods for mitigating cardiovascular disease risk in a subject are disclosed wherein the methods comprise regular routine consumption of nutraceutical compositions comprising high oleic acid canola oil and/or combinations of high oleic acid canola oil and conventional canola oil. The nutraceutical compositions may be in the form of a tablet, capsule, softgel, gelcap, liquid, powder or other suitable means of ingestion.

In another aspect, the present disclosure also pertains to functional food compositions useful for mitigating cardiovascular disease risk in an obese subject when consumed at regularly occurring intervals. The functional food compositions generally comprise high oleic acid canola oils in combination with one or more functional food substrates. Suitable functional food substrates are exemplified by, but not limited to the following: cereal, pasta, baked goods (for example, cookies, cakes, crackers and muffins), nutrition, snack or meal replacement bars (for example, energy bars and granola bars), smoothie beverages, dressings, mayonnaise, sauces, margarines, spreads, dips, potato chips, tortilla chips, cooking oils and sprays, and the like.

In another aspect of the present disclosure, nutraceutical compositions suitable for regular consumption for mitigating cardiovascular disease risk in an obese subject are disclosed. The nutraceutical compositions generally comprise high oleic acid canola oil in combination with a nutraceutical carrier. The nutraceutical composition may be in the form of, without limitation, tablets, capsules, softgels, gelcaps, liquids, lozenges, solutions or any other suitable means for consumption by a subject. Furthermore, the nutraceutical composition may be in solid, semi-solid or liquid form, and in unit dosage forms comprising an effective amount of high oleic acid canola oil. Suitable nutraceutical carriers are exemplified by, but not limited to the following: anti-adherents such as magnesium stearate; binders such as sugar alcohols, polysaccharides and disaccharides; coatings such as gelatin, synthetic polymers, shellac; diluents; preservatives; and flavouring and colouring agents.

As mentioned previously, the inventors observed that combinations of conventional canola oil with high oleic acid canola oil are also useful for preventing the development of cardiovascular disease risk factor in rats fed high-fat diets. However, conventional canola oil on its own was not able to prevent increases in isovolumetric relaxation time in rats fed high-fat diets. Similarly, treatment of obese prone rats with other edible oils, including soybean oil, conventional canola oil plus flax oil, and high linoleic safflower oil, also did not prevent the increase in isovolumic relaxation time of high-fat fed rats. Soybean oil is one of the most widely consumed cooking oils in the world, and has a fairly high polyunsaturated fatty acid content (approximately 54% linoleic acid and approximately 8% alpha-linoleic acid) and relatively low monounsaturated fatty acid content (approximately 23% oleic acid), with the remainder being saturated fatty acids (approximately 15%) (Table 1). Flax oil contains a mixture of fatty acids, and is particularly rich in polyunsaturated fatty acids namely 57% alpha-linoleic acid and 16% linoleic acid. Flax oil additionally comprises approximately 18% monounsaturated fatty acid content. Safflower oil contains a high linoleic acid content (approximately 75%), lower monounsaturated fatty acid content (approximately 14% oleic acid) and approximately 10% saturated fatty acid content (see Table 1).

Therefore, given the cardioprotective benefits of high oleic acid canola oil when combined with conventional canola oil, aspects of the present disclosure pertain to functional food compositions comprising blends of high oleic acid canola oil and conventional canola oil for mitigating cardiovascular disease risk. The present disclosure also pertains to nutraceutical compositions comprising blends of high oleic acid canola oil and conventional canola oil for regular consumption to mitigate cardiovascular disease risks.

In another aspect, methods for mitigating cardiovascular disease risk in a subject in need thereof are disclosed, wherein the functional food compositions of the present disclosure are provided to subjects for routine consumption on a regular basis.

In a further aspect, this disclosure pertains to methods for mitigating cardiovascular disease risk in subjects by their regular consumption of nutraceuticals as disclosed herein.

The aspects and embodiments of the present disclosure are further illustrated in the following examples.

### EXAMPLES

### Example 1

Five-week old selectively bred Obese-Prone (“OP”) rats were purchased from Charles River Laboratories International Inc. (St. Constant, QC, Canada). Animals were acclimatized in temperature and humidity-controlled rooms with a 12-h dark and 12-h light period cycle for one week prior to commencing delivery of high-fat diet regimens (“HF”). OP rats were separated into six groups, and each group was fed a selected HF diet regimen (energy from fat 55%, carbohydrate 30% and protein 15%) for a period of 12 weeks. The ingredient compositions of the HF diet formulations are shown in Table 2, while the fatty acid compositions of the HF diet formulations are shown in Table 3.

The diet regimens were refreshed twice per week. Food intake by the rats in each group, was monitored daily by weighing the food pans. All rats received tap water ad libitum. Body weights were determined weekly.

### General Characteristics.

Feed intake was assessed in all animals during the 12-week course of the study. At the end of the study, all animals were weighed and sacrificed. Adiposity (fat mass) and lipidemia (serum triglycerides, cholesterol and free fatty acids) (data not shown) were also measured. Hearts were removed, washed in ice-cold saline and their weights measured. Left ventricular tissue was separated, flash-frozen in liquid nitrogen and subsequently stored at −85° C. for further analyses.

### Assessment of Cardiac Structure and Function:

At the end of the 12-week dietary regimens, cardiac structure and functions were assessed by echocardiography following the procedures taught by Wojciechowski et al. (2010, Resveratrol arrests and regresses the development of pressure overload but not volume overload induced cardiac hypertrophy in rats. J. Nutr. 140(5):962-968).

TABLE 2
Dietary formulations.

| | Dietary compositions* | | | | | |
|---|---|---|---|---|---|---|
| g/kg diet | HC | C | CF | SF | SB | L |
| Cornstarch | 209 | 209 | 209 | 209 | 209 | 209 |
| Maltodextrin | 69.4 | 69.4 | 69.4 | 69.4 | 69.4 | 69.4 |
| Sucrose | 100 | 100 | 100 | 100 | 100 | 100 |
| Cellulose | 63.8 | 63.8 | 63.8 | 63.8 | 63.8 | 63.8 |
| Casein | 186.2 | 186.2 | 186.2 | 186.2 | 186.2 | 186.2 |
| High oleic acid | 308.3 | 0 | 0 | 0 | 0 | 0 |
| canola oil | | | | | | |
| Canola oil | 0 | 308.3 | 231.2 | 0 | 0 | 0 |
| Flaxseed oil | 0 | 0 | 77.1 | 0 | 0 | 0 |
| Safflower oil | 0 | 0 | 0 | 308.3 | 0 | 0 |
| Soybean oil | 0 | 0 | 0 | 0 | 308.3 | 28.5 |
| Lard | 0 | 0 | 0 | 0 | 0 | 279.8 |
| AIN-93G-MX^{b} | 44.6 | 44.6 | 44.6 | 44.6 | 44.6 | 44.6 |
| AIN-93-VX^{c} | 12.7 | 12.7 | 12.7 | 12.7 | 12.7 | 12.7 |
| L-Cystine | 3 | 3 | 3 | 3 | 3 | 3 |
| Choline | 3.2 | 3.2 | 3.2 | 3.2 | 3.2 | 3.2 |
| Bitartrate | | | | | | |
| Butylated | 0.037 | 0.037 | 0.037 | 0.037 | 0.037 | 0.037 |
| hydroxytoluene | | | | | | |

*HC = high-oleic acid canola oil C = conventional canola oil CF = mixture of conventional canola and flax oils SF = safflower oil SB = soybean oil L = lard
^{a}American Institute of Nutrition-93G mineral mix
^{b}American Institute of Nutrition-93G vitamin mix

TABLE 3
Fatty acid composition of the diet formulations

Diet*

| Fatty Acid^{a} | HC | C | CF | SF | SB | L |
|---|---|---|---|---|---|---|
| Total SFA | 7 | 7 | 8 | 10 | 15 | 49 |
| C16:0 | 4 | 4 | 4 | 6 | 10 | 24 |
| C18:0 | 2 | 2 | 2 | 3 | 4 | 21 |
| Total MUFA | 78 | 66 | 54 | 17 | 21 | 42 |
| C18:1 | 76 | 64 | 53 | 16 | 20 | 39 |
| Total PUFA | 16 | 27 | 38 | 73 | 63 | 9 |
| LA | 14 | 19 | 18 | 73 | 54 | 8 |
| ALA | 2 | 8 | 20 | 0.2 | 9 | 1 |
| LA/ALA | 7 | 2 | 1 | 365 | 6 | 8 |
| Total n-6 | 14 | 19 | 19 | 73 | 54 | 8 |
| Total n-3 | 2 | 8 | 20 | 0.4 | 9 | 1 |
| n-6/n-3 | 7 | 2 | 1 | 183 | 6 | 7 |

*HC = high-oleic acid canola oil C = conventional canola oil CF = conventional canola oil blended with flax oil SF = safflower oil SB = soybean oil L = lard
^{a}g/100 g fatty acids SFA = saturated fatty acids MUFA = mono-unsaturated fatty acids PUFA = poly-unsaturated fatty acids LA = linoleic acid ALA = alpha-linoleic acid

### Measurement of Cardiac Function In Vivo.

Two-dimensional-guided M-mode echocardiography and pulse-wave Doppler echocardiography were used to assess cardiac function. Contractile parameters of systolic function such as LV ejection fraction and cardiac output were assessed by 2D-guided M-mode echocardiography. Diastolic function was assessed by measuring the isovolumic relaxation time (IVRt) using pulse-wave Doppler echocardiography.

### Results.

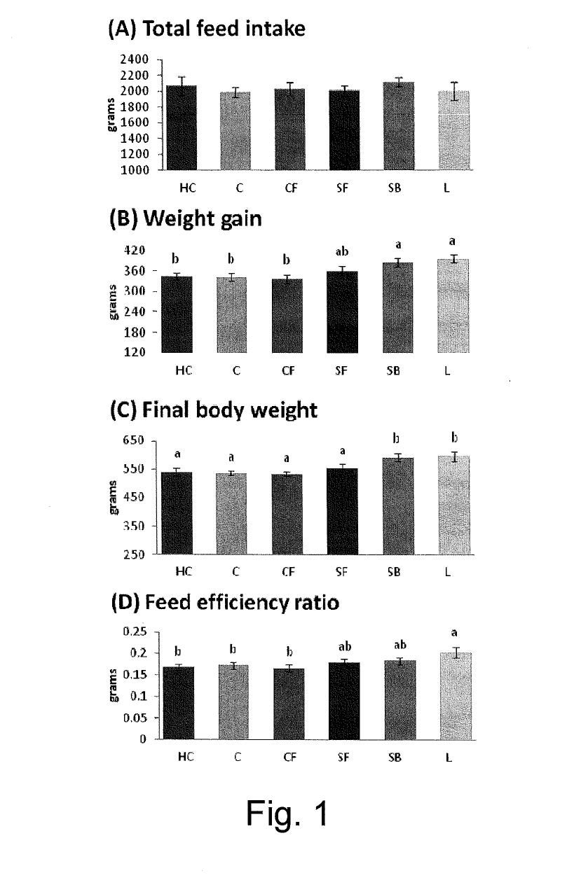

The OP rats were prepared and fed as provided above. During the course of the study, feed intake of all animals was assessed. With respect to total feed intake, there were no differences among groups receiving the different diet regimens (FIG. 1(A)). The groups fed the high oleic canola oil diet (HC), the conventional canola oil diet (C), and the diet comprising the blend of conventional canola oil and flax oil (C/F), gained the least amount of weight during the study (FIG. 1(B)) and had the lowest final body weights (FIG. 1(C)). The groups fed the soybean oil diet (SB) and lard diet (L) showed the greatest weight gains among the groups that gained the most amount of weight and had the highest final body weights (FIG. 1(C)). The group fed the safflower oil diet (SF) and the weight-marched control group (WM) fed the standard diet of PROLAB® RMH 3000 evidenced intermediate amounts of weight gain and final body weights (FIG. 1(C)). A feed efficiency ratio was calculated (total weight gain [g]/total feed intake [g]) to assess conversion of food mass into body mass. The group fed the lard diet (L) had the highest feed efficiency ratio while the group diets comprising the high oleic canola oil (HC), canola oil (C), and the blend of conventional canola oil and flax oil (C/F) had the lowest feed efficiency ratio (FIG. 1(D)). The other groups had intermediate feed efficiency ratios (FIG. 1(D)).

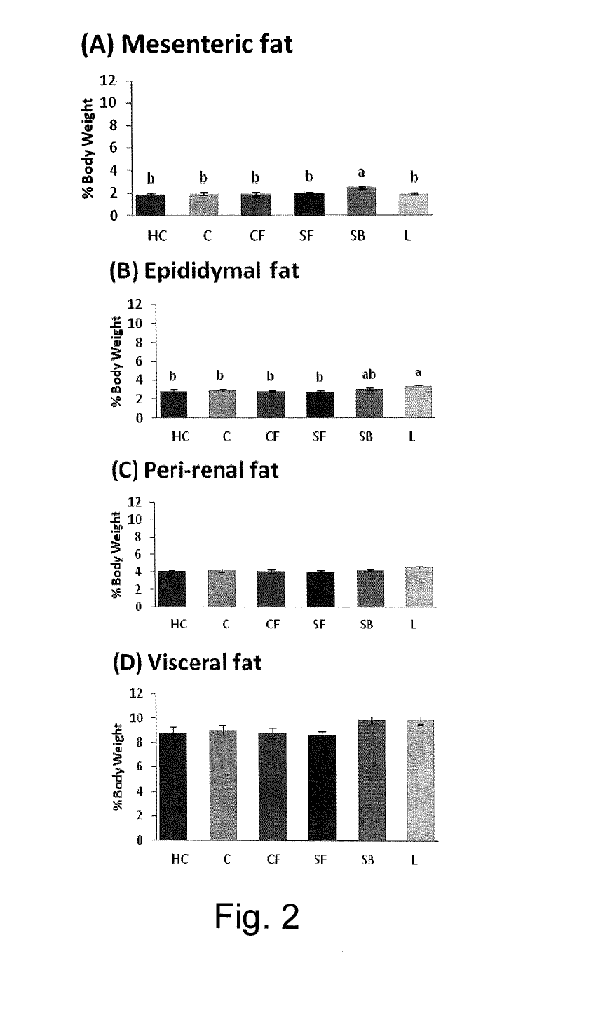

The weights of fat pads determined as percentages of body weights, were used as an indicator of obesity (FIG. 2). Groups fed the soybean oil diet (SB) had the highest mesenteric fat pads as a percentage of body weight compared to all other dietary groups (FIG. 2(A)). The weights of epididymal fat pads, peri-renal fat pads, and visceral fat pads, as percentages of body weights, were lower in high oleic canola (HC), canola (C), and the blend of conventional canola oil and flax oil (C/F) in comparison to the lard diet (L) (FIGS. 2(A), 2(B), 2(C), respectively).

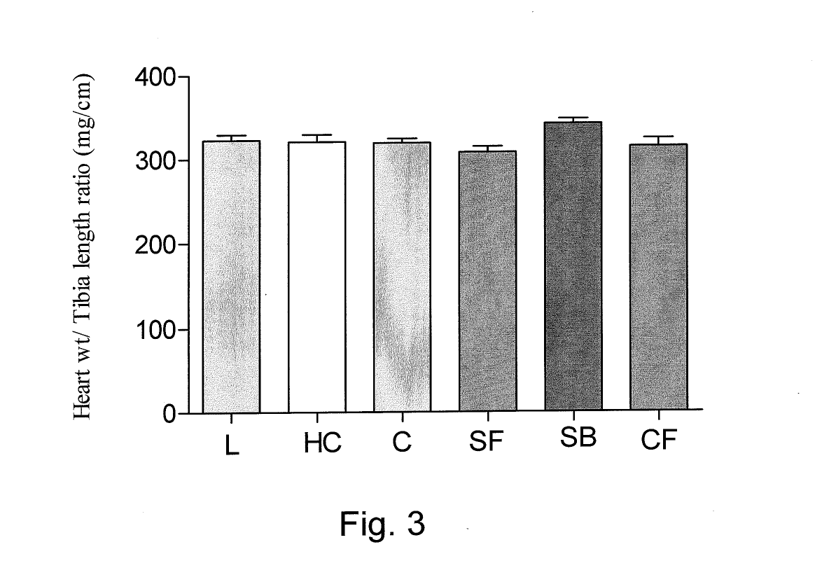

The weight heart to tibia length ratios (a marker for cardiac hypertrophy) of rats fed with high-fat dietary regimes did not differ significantly between any of the groups (FIG. 3).

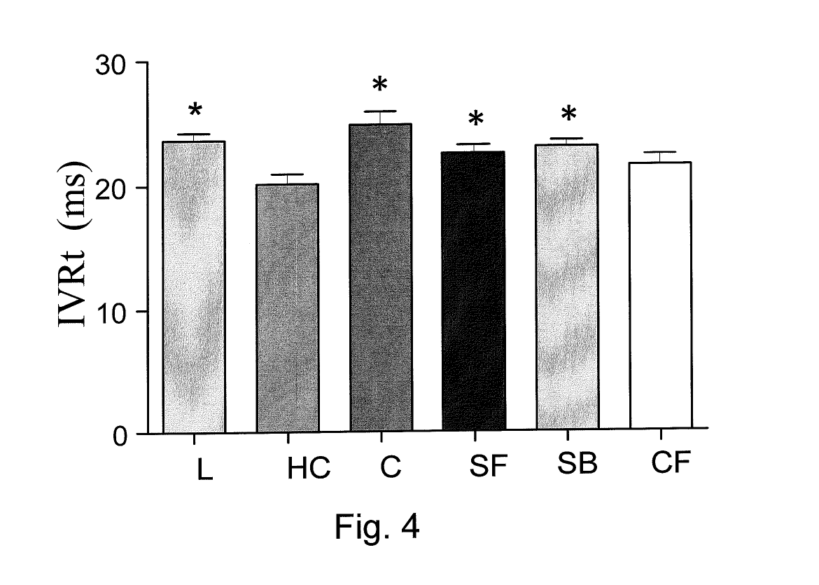

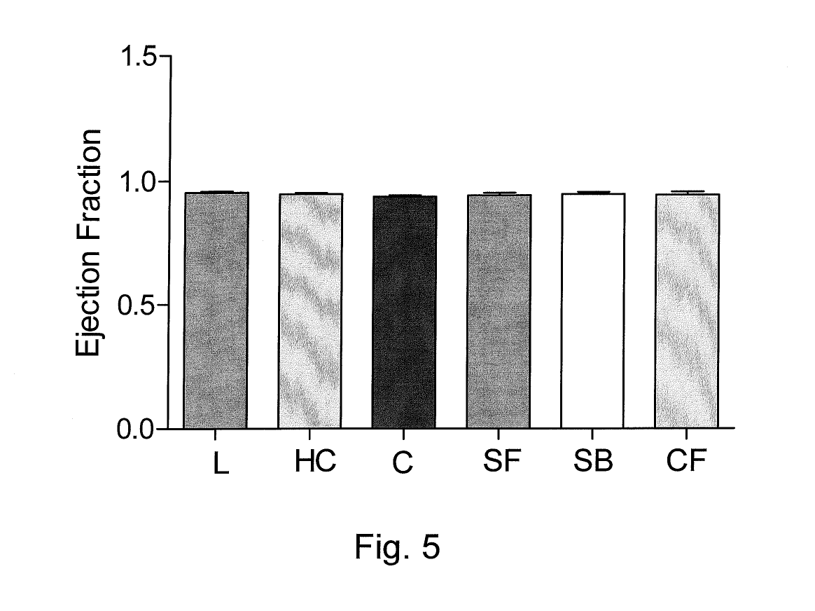

Isovolumic relaxation times (“IVRT”), a diastolic heart function parameter, were significantly higher (P≦0.05) in rats fed with high-fat dietary regimens comprising lard (L) or conventional canola oil (C) or safflower oil (SF) or soybean oil (SB) in comparison to the rats fed with high-fat diet regimens comprising high oleic acid canola oil (HC) or conventional canola oil blended with flax oil (C/F) (FIG. 4). However, ejection fraction, a systolic heart function parameter, was similar across all high-fat regimens (FIG. 5).

The results of this study indicate that obese-prone rats fed for 12 weeks with a high-fat diet comprising one of lard or conventional canola oil (C) or safflower oil (SF) or soybean oil (SB) evidenced significant impairments in the ability of their hearts to relax. However, obese-prone rats fed with a high-fat diet comprising high oleic acid canola oil (HC) or a blend of high oleic acid canola oil and conventional canola oil did not development the types of cardiac abnormalities noted with the other high-fat dietary regimens.

### Example 2

Five-week old selectively bred Obese-Prone (“OP”) rats and Sprague-Dawley (“SD”) rats were purchased from Charles River Laboratories International Inc. (St. Constant, QC, Canada). Animals were acclimatized in temperature and humidity-controlled rooms with a 12-h dark and 12-h light period cycle for one week prior to commencing delivery of high-fat (“HF”) diet regimens or standard diet regimens. OP rats were fed a HF diet comprising energy from fat 55%, carbohydrate 30% and protein 15% for a period of 12 weeks. The controls for this study were SD rats feed a low-fat diet. The ingredient compositions of the diet formulations are shown in Table 4, while the fatty acid compositions of the diet formulations are shown in Table 5.

Fresh diet was provided twice per week. Food intake was monitored daily by weighing the food pans. All rats received tap water ad libitum. Body weights were determined weekly.

### General.

At the end of the 12-week study, all animals were weighed and sacrificed. Adiposity (fat mass) and lipidemia (serum triglycerides, cholesterol and free fatty acids) (data not shown) were also measured. Hearts were removed, washed in ice-cold saline and their weights measured. Left ventricular tissue was separated, flash-frozen in liquid nitrogen and subsequently stored at −85° C. for further analyses.

TABLE 4
Diet Formulations.

| | High-fat diet fed to | Low-fat diet fed to |
|---|---|---|
| g/kg diet | OP rats | SD rats |
| Cornstarch | 209 | 347 |
| Maltodextrin | 69.4 | 115.3 |
| Sucrose | 100 | 165.7 |
| Cellulose | 63.8 | 50.2 |
| Casein | 186.2 | 155.5 |
| High oleic acid canola oil | 154.2 | 0 |
| Canola oil | 154.2 | 0 |
| Flaxseed oil | 0 | 0 |
| Safflower oil | 0 | 0 |
| Soybean oil | 0 | 116.5 |
| Lard | 0 | 0 |
| AIN-93G-MX^{a} | 44.6 | 35 |
| AIN-93-VX^{b} | 12.7 | 10 |
| L-Cystine | 3 | 2.3 |
| Choline Bitartrate | 3.2 | 2.5 |
| BHT | 0.006 | 0.006 |

^{a}American Institute of Nutrition-93G mineral mix
^{b}American Institute of Nutrition-93G vitamin mix

TABLE 5
Fatty Acid Composition of Diets

| | High-fat diet fed to | Low-fat diet fed to |
|---|---|---|
| Fatty Acid^{a} | OP rats | SD rats |
| Total SFA | 8 | 17 |
| C16:0 | 4 | 10 |
| C18:0 | 2 | 4 |
| Total MUFA | 65 | 21 |
| C18:1 | 60 | 19 |
| Total PUFA | 27 | 62 |
| LA | 18 | 52 |
| ALA | 8 | 9 |
| LA/ALA | 2 | 6 |
| Total n-6 | 19 | 53 |
| Total n-3 | 8 | 9 |
| n-6/n-3 | 2 | 6 |

^{a}g/100 g fatty acids
SFA = saturated fatty acids
MUFA = mono-unsaturated fatty acids
PUFA = poly-unsaturated fatty acids
LA = linoleic acid
ALA = alpha-linoleic acid

### Measurement of Cardiac Function In Vivo.

Two-dimensional-guided M-mode echocardiography and pulse-wave Doppler echocardiography were used to assess cardiac function. Contractile parameters of systolic function such as LV ejection fraction and cardiac output were assessed by 2D-guided M-mode echocardiography. Diastolic function was assessed by measuring the isovolumic relaxation time (IVRt) using pulse-wave Doppler echocardiography.

### Results.

The data in Table 6 show that obese-prone rats fed with a high-fat dietary regime comprising a blend of high-oleic acid canola oil and conventional canola oil (OP-HC+C), gained less weight over the 12-week treatment period compared to the “control” Sprague-Dawley rats fed with a low-fat dietary regime (SD-LF), but developed significantly more mesenteric fat. However, both groups of rats developed similar amounts of peri-renal fat and visceral fat (Table 6).

TABLE 6

Dietary formulation

| Parameter | OP & high-fat | SD & low-fat |
|---|---|---|
| Final body weight (g) | 552 ± 18 | 681 ± 39* |
| Mesenteric fat (g/100 g) | 2.1 ± 0.1 | 1.6 ± 0.2 |
| Epididymal fat (g/100 g) | 3.0 ± 0.1 | 3.3 ± 0.3 |
| Per-renal fat (g/100 g) | 4.7 ± 0.1 | 4.8 ± 0.6 |
| Visceral fat (g/100 g)^{a} | 9.8 ± 0.2 | 9.7 ± 1.1 |

^{a}Visceral fat = mesenteric fat + epididymal fat + per-renal fat
*P ≦ 0.05 v OP & high-fat

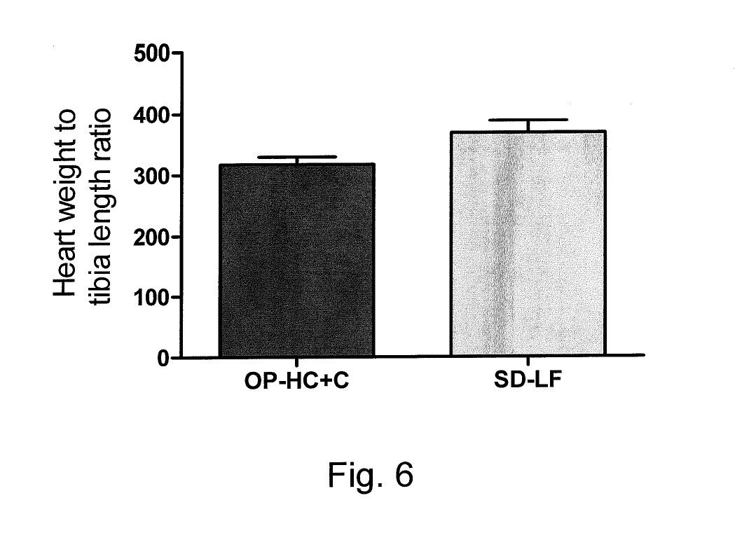

FIG. 6 shows the heart weight to tibia length ratios of: (i) obese-prone rats fed with a high-fat dietary regime comprising a blend of high-oleic acid canola oil and conventional canola oil (OP-HC+C), and (ii) the “control” Sprague-Dawley rats fed with a low-fat dietary regime (SD-LF).

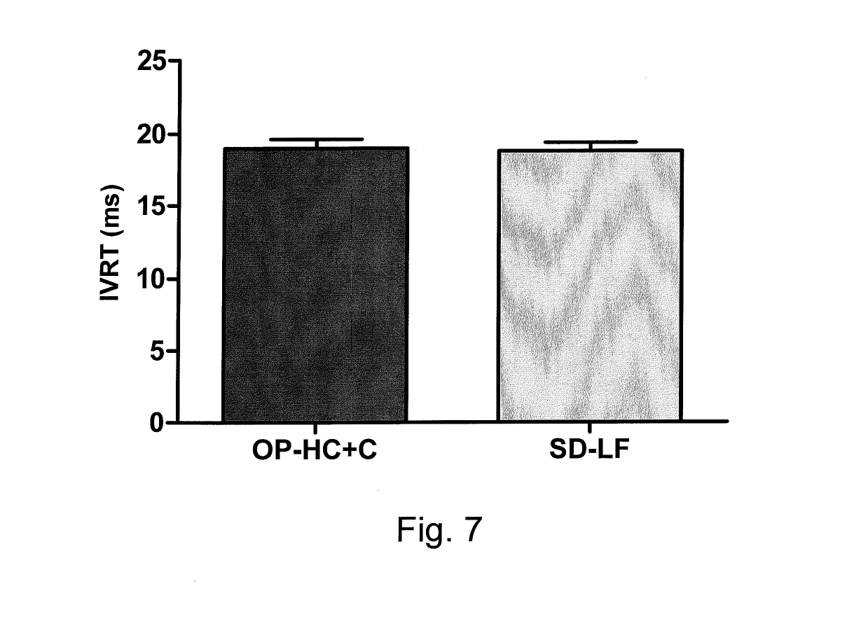

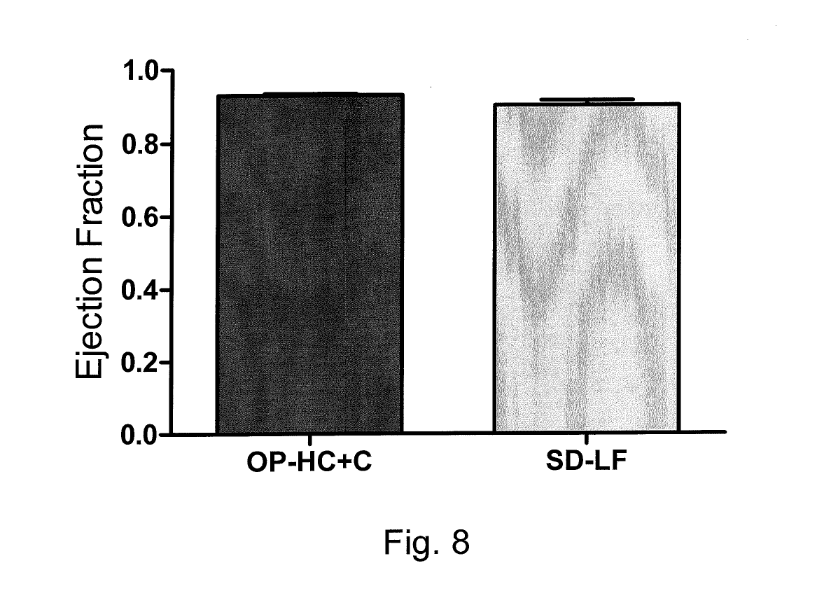

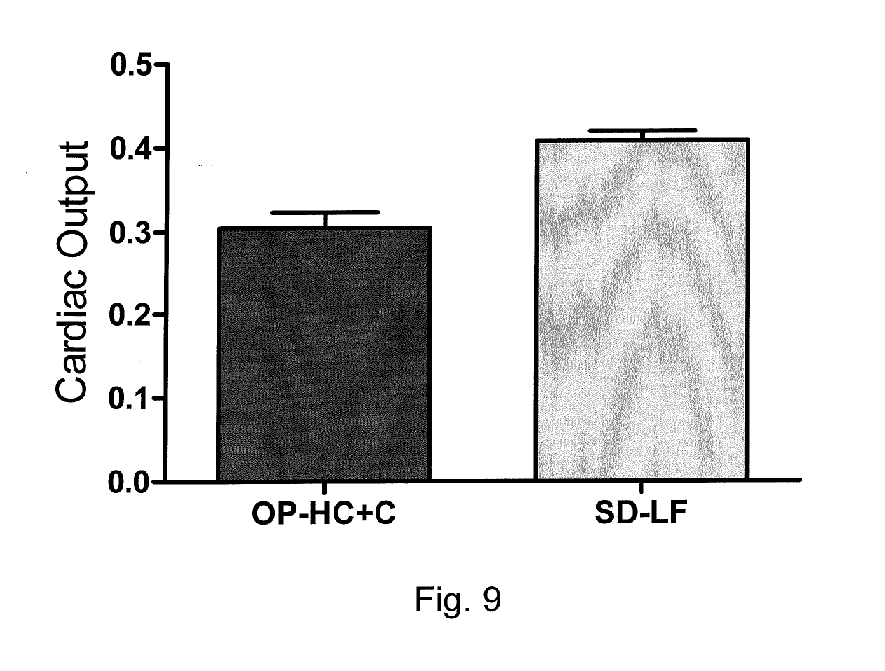

The data in FIG. 7 indicate that the IVRT was not significantly different for: (i) obese-prone rats fed with a high-fat dietary regime comprising a blend of high-oleic acid canola oil and conventional canola oil (OP-HC+C), and (ii) the “control” Sprague-Dawley rats fed with a low-fat dietary regime (SD-LF). The data in FIG. 8 indicate that the ejection fraction was not significantly different for these two treatments. The data in FIG. 9 indicate that the cardiac output of rats fed with a high-fat dietary regime comprising a blend of high-oleic acid canola oil and conventional canola oil (OP-HC+C) was significantly less than the cardiac output of rats fed with a low-fat dietary regime (SD-LF).

1. A dietary regimen for mitigating cardiovascular disease risk in a subject, the regimen comprising consumption of at least one of a functional food composition comprising high oleic canola oil, a dietary supplement comprising high oleic canola oil, and a nutraceutical composition comprising high oleic canola oil, whereby the subject consumes at least 14 mg/100 g of body weight every 24-hour diurnal period.

2. A functional food composition comprising at least 100 mg/g (w/w) high oleic acid canola oil for consumption according to the dietary regimen of claim 1.

3. A prescription for use of the functional food composition of claim 2, on a diurnal basis, to mitigate cardiovascular disease risk in a subject.

4. A dietary supplement composition comprising at least 100 mg/g (w/w) high oleic acid canola oil for consumption according to the dietary regimen of claim 1.

5. A prescription for use of the dietary supplement composition of claim 4, on a diurnal basis, to mitigate cardiovascular disease risk in a subject.

6. A nutraceutical composition comprising at least 100 mg/g (w/w) high oleic acid canola oil for consumption according to the dietary regimen of claim 1.

7. A prescription for use of the nutraceutical composition of claim 6, on a diurnal basis, to mitigate cardiovascular disease risk in a subject.
